(19) United States
(12) Patent Application Publication
(10) Pub. No.: US 2024/0057615 A1
(43) Pub. Date: Feb. 22, 2024

(54) BIOLOGICAL SEED-COATING AGENT (SCA) INCLUDING FERMENTATION BROTH OBTAINED BASED ON CO-CULTIVATION OF TRICHODERMA AND BACILLUS SEQUENTIALLY INOCULATED, AND PREPARATION METHOD THEREOF

(71) Applicant: SHANGHAI JIAO TONG UNIVERSITY, Shanghai (CN)

(72) Inventors: Jie CHEN, Shanghai (CN); Peng LIU, Shanghai (CN); Xinhua WANG, Shanghai (CN)

(73) Assignee: SHANGHAI JIAO TONG UNIVERSITY, Shanghai (CN)

(21) Appl. No.: 18/260,041

(22) PCT Filed: Jun. 22, 2022
(86) PCT No.: PCT/CN2022/100273

§ 371 Date: Jun. 30, 2023

(30) Foreign Application Priority Data
Jun. 23, 2021 (CN) 202110700561.2

(51) Int. Cl. A01N 63/38; C12N 1/20; C12N 1/14; C12N 3/00; A01N 63/22; A01N 61/02; A01N 43/22; A01N 63/50; A01N 43/38

(52) U.S. Cl. CPC A01N 63/38; C12N 1/205; C12N 1/145; C12N 3/00; A01N 63/22; A01N 61/02; A01N 43/22; A01N 63/50; A01N 43/38; C12R 2001/125

(57) ABSTRACT

A biological seed-coating agent (SCA) including a fermentation broth obtained based on co-cultivation of Trichoderma and Bacillus sequentially inoculated, and a preparation method thereof are provided. According to a principle of synthesis biology, Trichoderma that induces disease resistance and stress resistance and Bacillus that antagonizes pathogens and promotes crop growth are inoculated in stages for co-fermentation to prepare a microbial co-culture solution. The microbial co-culture solution is mixed with diatomaceous earth and brassinolide according to a specified ratio to prepare a biological SCA in a dosage form of a powder. The biological SCA includes high contents of spores and antagonistic and growth-promoting substances.

### CROSS REFERENCE TO THE RELATED APPLICATIONS

This application is the national phase entry of International Application No. PCT/CN2022/100273, filed on Jun. 22, 2022, which is based upon and claims priority to Chinese Patent Application No. 202110700561.2, filed on Jun. 23, 2021, the entire contents of which are incorporated herein by reference.

### TECHNICAL FIELD

The present disclosure relates to a technique for preparing a biological seed-coating agent (SCA) based on co-cultivation (co-fermentation) of Trichoderma and Bacillus and a use of the biological SCA. Specifically, a microbial co-culture solution is prepared mainly through co-fermentation, and then a biological SCA in a dosage form of a powder is prepared with diatomaceous earth to adsorb the microbial co-culture solution and brassinolide as an additive. This biological SCA has high contents of Trichoderma and Bacillus live spores, antimicrobial peptaibols, indoleacetic acid (IAA), or the like, and thus exhibits characteristics such as a strong ability to induce disease resistance of a crop and a strong ability to promote the growth of a crop.

### BACKGROUND

Chemical SCAs are agrochemical agents most commonly used in current food crop production in China to control soil-borne diseases and underground pests, promote the growth of crops, and improve stress resistance. With the implementation of the “pesticide and fertilizer double reduction” plan in agricultural production in China, SCAs need to be eco-friendly and ecological. It has been proved by practice that microbial SCAs are an important way to provide seed probiotics. Microbial SCAs can reduce the harm of traditional agrochemicals to soil, and can also improve a microbial community structure in soil for a crop and promote the growth of a crop. However, the current microbial SCAs mainly have problems such as single effective microbial strains, poor spore stress resistance and activity, insufficient microbial metabolite utilization and unstable performance of plant disease prevention and crop growth-promoting effects. Therefore, the development and utilization of a multi-microbes SCA is of great significance for the improvement of the quality and efficacy of a biological SCA.

As one of the internationally-recognized biocontrol fungi, Trichoderma exhibits excellent prevention and restoration effects for soil-borne diseases and agricultural soil pollution. Trichoderma can produce biological disease control-associated substances such as hydrophobic proteins, chitinases, dextranases, proteases, and antagonistic secondary metabolites (Chen Jie, Progress of Research on Induction of Trichoderma for Disease Resistance of Plants [J], Chinese Journal of Biological Control, 2014, 31 (5): 733-741), and a single-Trichoderma biological SCA has been developed internationally (Nayaka S. C, et al. Seed biopriming with a novel strain of Trichoderma harzianum for the control of toxigenic Fusarium verticillioides and fumonisins in maize, Archiv für Pflanzenschutz. 2010, 264-282), which is mainly used in corn production. As one of the internationally-recognized biocontrol bacteria, Bacillus is mainly used for biological control of leaf diseases and growth promotion and can secrete antagonistic substances such as proteins, lipopeptides, and secondary metabolites thereof and growth-promoting substances such as IAA and indolebutyric acid (IBA). Currently, microbial agents of Trichoderma or Bacillus alone are commonly used in the control of crop diseases and promotion of crop growth but gradually exhibit shortcomings in use. For example, a microbial agent of Trichoderma alone shows an obvious control effect for soil-borne diseases, but has poor stress resistance; and a microbial agent of Bacillus alone has an obvious control effect for leaf diseases and strong stress resistance, but exhibits a weaker ability to induce crop resistance against disease and environmental adverse factors than Trichoderma. Therefore, in many manufacturers inside and outside China, Trichoderma and Bacillus are separately used to conduct fermentation, and then resulting fermentation broths are mixed to prepare a composite preparation, but it still cannot fully solve the problem of product homogeneity with Trichoderma and Bacillus inside and outside China. With the development of synthetic biology, the Trichoderma/Bacillus co-fermentation technology has become a research hotspot. Trichoderma and Bacillus interact with each other during a co-fermentation process to induce the production of a series of novel primary and secondary metabolites which is usually lacked in single culture of either one, those novel metabolites provide a material basis for the preparation of novel biological SCAs. Therefore, the research on a technique for preparing a novel biological SCA based on co-fermentation with Trichoderma and Bacillus is of great significance for the improvement of an innovation level of biological SCAs in China.

### SUMMARY

An objective of the present disclosure is to provide a method for preparing a biological SCA based on co-cultivation of Trichoderma and Bacillus, and a use method of the biological SCA. A technical focus of the present disclosure is as follows: A screened Trichoderma and Bacillus affinity combination is used to conduct co-fermentation by an established sequential inoculation method to obtain a fermentation broth with high contents of Trichoderma and Bacillus spores, antimicrobial peptaibols (such as alamethicin), and growth-regulating substances (such as IAA), and then a two-microbes co-culture SCA is prepared through adsorption of diatomaceous earth, thereby improving effects of the biological SCA to promote crop growth and control diseases.

The present disclosure is implemented by the following technical solutions:
1. A method for preparing a biological SCA based on co-cultivation of Trichoderma and Bacillus is provided, including the following steps:
S1: preparing a fermentation broth obtained based on co-cultivation of Trichoderma and Bacillus;
S2: mixing the fermentation broth obtained based on co-cultivation of Trichoderma and Bacillus with diatomaceous earth and brassinolide; and
S3: detecting a quality of the biological SCA in a dosage form of a powder.
2. In S1, a co-fermentation technique is used to increase an interaction metabolism level of Trichoderma and Bacillus while maintaining comparable numbers of spore of the two types of microbes.

As a preferred embodiment, in S1, a preparation method of the fermentation broth includes:
inoculating Trichoderma into a potato dextrose agar (PDA) medium, cultivating at 28° C. for 5 d, punching with a 5 mm puncher, inoculating a resulting Trichoderma disc into a potato dextrose (PD) medium, and cultivating in the PD medium for 3 d; transfer a resulting Trichoderma seed culture into a co-culture medium in a fermentation tank, and conducting fermentation at 28° C. for 48 h, with a ventilation of 0.8 vvm to 1 vvm, a pH of 5.0 to 5.6, and a rotational speed of 120 rpm;
inoculating the Bacillus into a lysogeny broth agar (LBA) medium, cultivating at 30° C. for 2 d, punching with a 5 mm puncher, inoculating a resulting Bacillus disc into a lysogeny broth (LB) medium, and cultivating at 30° C. for 2 d; and inoculating a resulting Bacillus seed culture into a Trichoderma fermentation broth (co-culture medium) obtained after 2 d of fermentation, and conducting co-fermentation with a ventilation of 1 vvm to 1.3 vvm, a pH adjusted by ammonia water to 4.5 to 5.5, and a rotational speed of 140 rpm. During fermentation, according to a co-fermentation time range of 45 h to 50 h, when a Bacillus spore formation rate is 85%, a Trichoderma chlamydospore formation rate is 90%, an antimicrobial peptaibol alamethicin content reaches 0.1 μg/L to 0.5 μg/L, and an IAA content reaches 2,000 μg/L to 2,500 μg/L, fermentation is completed, and a resulting fermentation broth is discharged.

As a preferred embodiment, in S2, the fermentation broth obtained based on co-cultivation of Trichoderma and Bacillus, diatomaceous earth, and brassinolide are mixed and dried as follows:
after the co-fermentation is completed, separating enriched mixed spores through solid-liquid separation (SLS) by a centrifuge, thoroughly mixing the enriched mixed spores, diatomaceous earth, and 0.1% brassinolide according to a mass ratio of 25%:60%:15%, oven-drying a resulting mixture in a spore dryer (at 45° C. to 48° C.) until a moisture content is 5%, and sieving a resulting dried material through an 80-mesh sieve to obtain a Trichoderma-Bacillus-brassinolide dry-powder biological SCA.

As a preferred embodiment, in S3, a dilution-plate method is used to detect the microbial co-culture powder, and detection results are as follows: Trichoderma spore content: 0.8×10⁸ cfu/g to 1.5×10⁸ cfu/g, Bacillus spore content: 2.0×10⁹ cfu/g to 5.0×10⁹ cfu/g, antimicrobial peptaibol (alamethicin) content: 0.1039 μg/L, and IAA content: 2159.53 μg/L.

### BRIEF DESCRIPTION OF THE DRAWINGS

FIG. 1 shows the influence of SCAs on a wheat emergence rate; and

FIG. 2 shows the control effects of SCAs obtained based on co-cultivation of Trichoderma and Bacillus for Fusarium head blight (FHB).

### DETAILED DESCRIPTION OF THE EMBODIMENTS

The present disclosure is described in detail below with reference to specific examples. The following examples will help those skilled in the art to further understand the present disclosure, but do not limit the present disclosure in any way. It should be noted that those of ordinary skill in the art can further make several variations and improvements without departing from the idea of the present disclosure. These all fall within the protection scope of the present disclosure.

### Example 1

A biological SCA obtained based on co-fermentation of Trichoderma and Bacillus was prepared, and specific steps were as follows:

1. Plate cultivation of Trichoderma and Bacillus: Trichoderma was inoculated into a PDA medium and cultivated at 28° C. for 3 d. Bacillus was inoculated into an LBA medium and cultivated at 30° C. for 2 d.

PDA medium: 200 g of a potato was taken, peeled, diced, and then steamed; a resulting supernatant was taken, 20 g of glucose and 20 g of an agar powder were added, and a resulting mixture was diluted with deionized water to 1 L, dispensed into 250 mL erlenmeyer flasks, and autoclaved at 121° C. for 30 min.

PD medium: 200 g of a potato was taken, peeled, diced, and then steamed; a resulting supernatant was taken, 20 g of glucose was added, and a resulting mixture was diluted with deionized water to 1 L, dispensed into 250 mL erlenmeyer flasks, and autoclaved at 121° C. for 30 min.

LBA medium: 5 g of a yeast extract, 10 g of sodium chloride, 10 g of tryptone, and 20 g of an agar powder were mixed, a resulting mixture was diluted with deionized water to 1 L and dispensed into 250 mL erlenmeyer flasks, a pH was adjusted with NaOH to 7.0, and a resulting medium was autoclaved at 121° C. for 20 min.

LB medium: 5 g of a yeast extract, 10 g of sodium chloride, and 10 g of tryptone were mixed, a resulting mixture was diluted with deionized water to 1 L and dispensed into 250 mL erlenmeyer flasks, a pH was adjusted with NaOH to 7.0, and a resulting medium was autoclaved at 121° C. for 20 min.

2. Preparation of a Trichoderma-Bacillus co-fermentation medium: 20 g/L corn flour, 20 g/L yeast extract powder, and 20 g/L molasses were mixed, and a pH of a resulting medium was adjusted with sodium hydroxide to 6.0 to 6.5.

3. Control of a sequential inoculation and co-fermentation process: In a 300 L fermentation tank, a fermentation medium was sterilized at 121° C. for 30 min; after the sterilization was completed, a Trichoderma seed culture was inoculated into a co-culture medium (1% v/v), and fermentation was conducted for 40 h with a ventilation of 1 vvm, a rotational speed of 180 rpm, a temperature of 28° C., and a pH adjusted by ammonia water to 4.5 to 5.5, a Bacillus seed culture was inoculated into a resulting fermentation broth (2.0% v/v), and co-fermentation was conducted for 48 h; and when a Bacillus spore formation rate reached 85% and a Trichoderma chlamydospore formation rate reached 90%, a resulting fermentation broth was discharged.

4. Detection of a quality of the co-fermentation: Contents of IAA and alamethicin in each of a Trichoderma-Bacillus co-fermentation supernatant and a Trichoderma fermentation supernatant were detected. In the co-fermentation supernatant, an alamethicin content was 0.1039 μg/L and an IAA content was 2159.53 μg/L, which were significantly higher than those in a supernatant obtained after fermentation of Trichoderma alone and a supernatant obtained after fermentation of Bacillus alone; and contents of the antimicrobial peptaibol (alamethicin) and IAA in a co-fermentation broth were significantly higher than those in a single-strain fermentation broth (as shown in Table 1).

5. Preparation of a dry-powder SCA based on co-cultivation of Trichoderma and Bacillus After the co-fermentation was completed, enriched mixed spores were separated through SLS by a centrifuge, the enriched mixed spores, diatomaceous earth, and 0.1% brassinolide were thoroughly mixed according to a mass ratio of 3:6:1, oven-dried in a spore dryer (at 45° C.) until a moisture content was 5%, and sieved through an 80-mesh sieve to obtain a Trichoderma-Bacillus-brassinolide dry-powder biological SCA.

TABLE 1
Analysis of contents of alamethicin and IAA in a supernatant obtained
after co-fermentation of Trichoderma and Bacillus and a supernatant
obtained after fermentation of Trichoderma alone

| | Component | Component content, |
|---|---|---|
| Fermentation type | type | μg/L |
| Co-fermentation of Trichoderma and | IAA | 2159.53 |
| Bacillus | Alamethicin | 0.1039 |
| Fermentation of Trichoderma alone | IAA | 23.82 |
| | Alamethicin | 0.0865 |
| Fermentation of Bacillus alone | IAA | 620.45 |
| | Alamethicin | 0.0120 |

### Example 2

A growth-promoting effect of the biological SCA obtained based on co-cultivation of Trichoderma and Bacillus was detected in the field.

Test site: Henan Academy of Agricultural Sciences, Zhumadian

Test Steps:

1. Preparation of field experimental materials

Variety: Jimai 22

Biological SCA: A dry-powder SCA obtained based on co-cultivation of Trichoderma and Bacillus

Chemical SCAs: 90% thiamethoxam, 8.5% tebuconazole, and a 1.5% film-forming agent (control)

2. The chemical SCAs (agent-to-seed ratio: 1:200) and the dry-powder SCA obtained based on co-cultivation of Trichoderma and Bacillus (agent-to-seed ratio: 1:100) each was used for seed dressing.

3. 20 d after the seeds were sown, the influence of each treatment on wheat emergence was investigated.

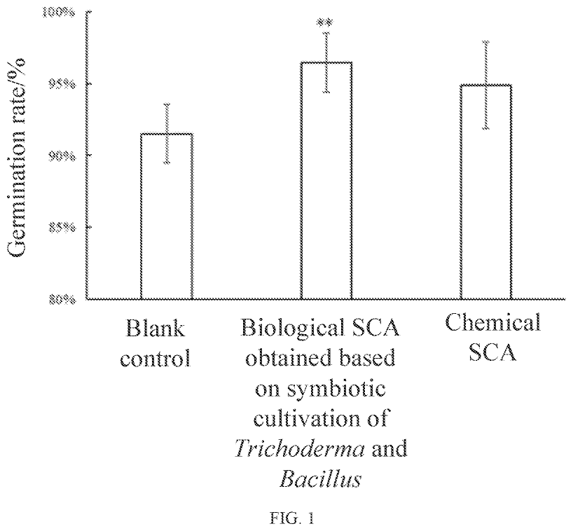

4. There was a very significant difference in emergence rate between the biological SCA and the blank control; and the influence of the biological SCA on a wheat emergence rate was similar to or better than the influence of the chemical SCAs on a wheat emergence rate (as shown in FIG. 1, it should be noted that data in FIG. 1 were an average of 5 replicates and a standard error thereof. **: one-way analysis of variance (ANOVA) indicated a very significant difference, 0.01<P<0.001).

### Example 3

A control effect of the dry-powder SCA obtained based on co-cultivation of Trichoderma and Bacillus for soil-borne diseases of corn was verified in the field.

Test site. A farm of Shenyang Agricultural University

Test Steps:

1. Field experimental materials:

2. Variety: Zhengdan 958

3. Biological SCA: A dry-powder SCA obtained based on co-cultivation of Trichoderma and Bacillus, which was used with an agent-to-seed ratio of 1:100.

4. Chemical SCAs: 90% thiamethoxam, 8.5% tebuconazole, and a 1.5% film-forming agent, which each was used with an agent-to-seed ratio of 1:200.

5. Sowing mode: Dibbling, 4,000 plants/mu.

6. Pathogen inoculation: When sowing, Fusarium graminearum (F graminearum) and Rhizoctonia solani (R. solani) each was inoculated into the soil at an inoculum size of 5%, in mid-to-late July, a lower leaf sheath was inoculated by the wheat grain-cultured R solani; and in early August, a fruit ear was inoculated with Fusarium verticilloides (F. verticilloides) by a toothpick method.

Control Effect Investigation and Production Test.

1. At a milk ripening stage, 5 diagonal points were investigated for each treatment, 4 rows were randomly selected for each point, and 50 plants were randomly investigated for each row. A disease incidence (including a lodging rate) of each treatment was investigated, and a field control effect of stalk rot was statistically analyzed.

2. At a milk ripening stage, 5 diagonal points were investigated for each treatment, 4 rows were randomly selected for each point, and 50 plants were randomly investigated for each row. A disease incidence and a disease level per plant for each treatment were investigated, and a disease index and a field control effect for corn sheath blight were statistically analyzed.

Disease indexes of corn sheath blight were shown in Table 2.

TABLE 2
Grading criteria for com sheath blight

| Disease | |
|---|---|
| rank | Symptom description |
| 1 | The 4th leaf sheath under a fruit ear and |
| | lower leaf sheaths are diseased. |
| 3 | The 3rd leaf sheath under a fruit ear |
| | and lower leaf sheaths are diseased. |
| 5 | The 2nd leaf sheath under a fruit ear and |
| | lower leaf sheaths are diseased. |
| 7 | The 1st leaf sheath under a fruit ear and |
| | lower leaf sheaths are diseased |
| 9 | A fruit ear and upper leaf sheaths are diseased. |

3. At a milk ripening stage, 4 rows of each treatment were randomly selected, and 10 ears were randomly selected from each row. Bracts were peeled off, a disease level of each ear was investigated and recorded, and a disease index and a control effect for the ear were statistically analyzed. Grading criteria for disease incidence of corn ears were shown in Table 3.

TABLE 3
Grading criteria for corn ear rot

| Disease rank | Symptom description |
|---|---|
| 1 | A diseased area is 0% to 1% of a total area of a fruit ear. |
| 3 | A diseased area is 2% to 10% of a total area of a fruit ear. |
| 5 | A diseased area is 11% to 25% of a total area of a fruit ear. |
| 7 | A diseased area is 26% to 50% of a total area of a fruit ear. |
| 9 | A diseased area is 51% to 100% of a total area of a fruit |
| | ear. |

4. Yield Measurement Method

At a harvest stage, 5 points were selected for each treatment to investigate 10 m² of plants. 10 to 20 plants were selected from each point and investigated, and a number of corn ears, a thousand-grain weight, and a moisture content in grains at harvest each was measured. A yield of each plot was recorded, a yield per acre was calculated, and a yield increase rate in contrast to a blank control (in the blank control, neither the dry-powder SCA obtained based on co-cultivation of Trichoderma and Bacillus or the chemical SCA was added) was calculated.

5. Field test results showed that the dry-powder SCA obtained based on co-cultivation of Trichoderma and Bacillus could effectively reduce the occurrence of stalk rot and significantly increase the yield (as shown in Table 4).

TABLE 4
Corn stalk rot control effect and yield increase effect of the dry-powder
SCA obtained based on co-cultivation of Trichoderma and Bacillus

| | | | Number | | | |
|---|---|---|---|---|---|---|
| | | Control | of | | | Yield |
| | Incidence | effect | ears/10 | Thousand- | Yield | increase |
| Treatment | (%) | (%) | m² | grain weight/g | kg/mu | rate (%) |
| Dry-powder | 0.93 ± 0.46a | 74.86 | 53.67a | 372.80 ± 15.55a | 736.80 ± 9.72a | 19.28 |
| SCA | | | | | | |
| obtained | | | | | | |
| based on co- | | | | | | |
| cultivation | | | | | | |
| of | | | | | | |
| Trichoderma | | | | | | |
| and Bacillus | | | | | | |
| Chemical | 1.39 ± 0.80a | 62.43 | 54.33a | 350.10 ± 31.65a | 712.95 ± 3.11a | 15.42 |
| SCA | | | | | | |
| Blank | 3.70 ± 0.46b | — | 53.33a | 317.10 ± 31.65a | 617.70 ± 9.95b | — |
| control | | | | | | |

The dry-powder SCA obtained based on co-cultivation of Trichoderma and Bacillus exhibited an excellent control effect for corn sheath blight and an excellent yield increase effect (as shown in Table 5 and Table 6).

TABLE 5
Corn sheath blight control effect and yield increase effect of the dry-powder
SCA obtained based on co-cultivation of Trichoderma and Bacillus (plot 1)

| | | | Number | | | |
|---|---|---|---|---|---|---|
| | | Control | of | | | Yield |
| | Disease | effect | ears/10 | Thousand- | Yield | increase |
| Treatment | index | (%) | m² | grain weight/g | kg/mu | rate (%) |
| Dry-powder | 33.81 ± 5.67ab | 48.80 | 59.33a | 352.67 ± 7.68a | 746.50 ± 11.33a | 10.31 |
| SCA | | | | | | |
| obtained | | | | | | |
| based on co- | | | | | | |
| cultivation | | | | | | |
| of | | | | | | |
| Trichoderma | | | | | | |
| and Bacillus | | | | | | |
| Chemical | 42.01 ± 1.60b | 36.38 | 58.33a | 349.23 ± 19.83a | 720.15 ± 3.11a | 6.43 |
| SCA | | | | | | |
| Blank | 66.03 ± 3.06c | — | 56.33a | 317.03 ± 16.78a | 676.73 ± 10.51a | — |
| control | | | | | | |

The SCA obtained based on co-cultivation of Trichodermna and Bacillus could improve the ear rot control effect and yield increase rate (Table 6).

TABLE 6
Corn sheath blight control effect and yield increase effect of the dry-powder
SCA obtained based on co-cultivation of Trichoderma and Bacillus (plot 2)

| | | | Number | | | |
|---|---|---|---|---|---|---|
| | | Control | of | | | Yield |
| | Disease | effect | ears/10 | Thousand- | Yield | increase |
| Treatment | index | (%) | m² | grain weight/g | KG/667 m² | rate (%) |
| Dry-powder | 23.81 ± 0.58a | 36.86 | 59.37a | 363.67 ± 9.14a | 757.94 ± 9.40a | 16.93 |
| SCA | | | | | | |
| obtained | | | | | | |
| based on co- | | | | | | |
| cultivation | | | | | | |
| of | | | | | | |
| Trichoderma | | | | | | |
| and Bacillus | | | | | | |
| Chemical | 26.06 ± 1.65a | 30.89 | 57.67a | 352.67 ± 14.50a | 726.67 ± 8.39a | 12.10 |
| SCA | | | | | | |
| Blank | 37.71 ± 0.58b | — | 57.67a | 328.83 ± 8.20a | 648.21 ± 7.67b | — |
| control | | | | | | |

### Example 4

Control effects of SCAs obtained based on co-cultivation of Trichoderma and Bacillus for FHB

1. Test site: A farm of Shanghai Jiao Tong University (Minhang Campus)

Test materials: Bacillus subtilis (B. subtilis) BS-22, Trichoderma aureoviride (T. aureoviride) SG3403, F. graminearum, Rhizoctonia solani, and wheat variety Ningmai 13.

2. Preparation of dry-powder and liquid SCAs: A microbial co-culture solution, a film-forming agent, and water were mixed in a ratio of 8:1:1 to obtain a liquid SCA. A microbial co-culture solution and talcum powder were mixed in a ratio of 1:5 (ml:g) and then oven-dried in a 40° C. oven for 5 h to 6 h to obtain a dry-powder SCA.

3. F. graminearum was cultivated in a shaking flask for about 7 d (2×10⁸ cfu/L).

4. Pot soil was prepared with an organic seedling medium and vermiculite in a ratio of 3:1 (V/V). The prepared pot soil was filled into flower pots (high×diameter: 27 cm×23 cm). Then coated wheat seeds (Ningmai 13) were sown, with 10 seeds per pot and a covered soil thickness of 1 cm.

5. 14 d to 21 d after sowing, three wheat plants were randomly selected from each pot and then tested for the plant height and fresh weight. At a wheat flowering stage, an F graminearum spore suspension inoculum was diluted 50-fold (1×10⁷ cfu/L) and then sprayed onto wheat plants, and the wheat plants were highly moisturized with a humidifier. 20 d later, the incidence of FHB was observed, and the disease index and control effect were investigated. FIG. 1 shows the influence of treatment of different SCAs on a wheat emergence rate.

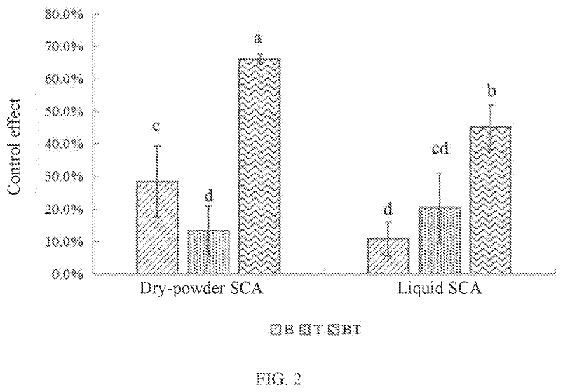

6. FIG. 2 shows the control effects of SCAs obtained based on co-cultivation of Trichoderma and Bacillus for FHB. It can be seen from FIG. 2 that control effects of treatments with a microbial agent obtained after fermentation of Bacillus alone, a microbial agent obtained after fermentation of Trichoderma alone, and the dry-powder SCA obtained after co-fermentation of Trichoderma and Bacillus reach 28.4%, 13.4%, and 66.2%, respectively (namely, the bars d, cd, and b for the dry-powder SCA in the figure), indicating that a control effect of the treatment with the dry-powder SCA for FHB is significantly higher than that of other treatments; and

control effects of treatments with a microbial agent obtained after fermentation of Bacillus alone, a microbial agent obtained after fermentation of Trichoderma alone, and the liquid SCA obtained after co-fermentation of Trichoderma and Bacillus reach 10.9%, 20.4%, and 45.2%, respectively (namely, the bars c, d, and a for the liquid SCA in the figure), indicating that a control effect of the treatment with the liquid SCA for FHB is significantly higher than that of other treatments.

It should be noted that B in FIG. 2 represents a microbial agent obtained after fermentation of Bacillus alone; T represents a microbial agent obtained after fermentation of Trichoderma alone; and BT represents an SCA obtained after co-fermentation of Trichoderma and Bacillus. For significance differences of the above control effects: P<0.05.

Compared with the prior art, one or more embodiments of the present disclosure have the following beneficial effects.

1. A fermentation broth obtained after co-fermentation of Trichoderma and Bacillus has higher contents of alamethicin and IAA than a fermentation broth obtained after fermentation of Trichoderma alone. It should be noted that, unlike the co-cultivation used in the prior art, co-fermentation is adopted in the present disclosure, which can overcome an antagonism or competition effect between microorganisms during co-cultivation to cause growth of a specified microorganism.

2. The dry-powder SCA obtained based on co-cultivation of Trichoderma and Bacillus has a more stable spore content than the liquid SCA.

3. The dry-powder SCA obtained based on co-cultivation of Trichoderma and Bacillus has more excellent functions of controlling diseases and promoting crop growth than a microbial agent obtained based on fermentation of Trichoderma or Bacillus alone and a simple combination of microbial agents obtained based on separate fermentation of the two.

The present disclosure is not limited to the above specific implementations, and a person skilled in the art can make various variations or modifications within the scope of the claims without affecting the essence of the present disclosure.

The specific examples of the present disclosure are described above. It should be understood that the present disclosure is not limited to the above specific implementations, and a person skilled in the art can make various variations or modifications within the scope of the claims without affecting the essence of the present disclosure. The examples of the present disclosure and features in the examples may be arbitrarily combined with each other in a non-conflicting situation.

What is claimed is:

1. A fermentation broth obtained based on a co-cultivation of Trichoderma and Bacillus sequentially inoculated, wherein the Thrichoderma is first inoculated at an amount 1% v/v to 2% v/v of a total fermentation amount and cultivated at 25° C. to 28° C. for 35 h to 45 h, then the Bacillus is inoculated at an amount 2.0% v/v to 3.0% v/v of the total fermentation amount, and the Trichoderma and the Bacillus are co-cultivated for 48 h at 28° C. to 30° C.

2. A biological seed-coating agent (SCA), comprising the fermentation broth obtained based on the co-cultivation of the Trichoderma and the Bacillus sequentially inoculated according to claim 1.

3. The biological SCA according to claim 2, wherein in the fermentation broth obtained based on the co-cultivation of the Trichoderma and the Bacillus, a content of a marker alamethicin is 0.04 μg/L to 0.05 μg/L, a content of indoleacetic acid (IAA) is 900 μg/L to 1,000 μg/L, a content of a Trichoderma spore is 1.0×10⁸ cfu/L to 9.0×10⁸ cfu/L, and a content of a Bacillus spore is 1.0×10⁹ cfu/L to 9.0×10⁹ cfu/L.

4. The biological SCA according to claim 2, wherein the biological SCA is in a dosage form of a dry powder.

5. The biological SCA according to claim 2, comprising: 20% w/w to 30% w/w of microbial biomass obtained after the co-cultivation of the Trichoderma and the Bacillus, 40% w/w to 60% w/w of a diatomaceous earth, and 12% w/w to 20% w/w of brassinolide;
wherein the brassinolide is in a form of a 0.1% solution.

6. A preparation method of the biological SCA according to claim 2, comprising the following steps:
S1: preparing the fermentation broth obtained based on the co-cultivation of the Trichoderma and the Bacillus;
S2: mixing the fermentation broth obtained based on the co-cultivation of the Trichoderma and the Bacillus with a diatomaceous earth and brassinolide; and
S3: detecting a quality of the biological SCA in a dosage form of a powder.

7. The preparation method of the biological SCA according to claim 6, wherein the Trichoderma is Trichoderma aureoviride (T. aureoviride) SG3403, and the Bacillus is Bacillus subtilis (B. subtilis) B22.

8. The preparation method of the biological SCA according to claim 6, wherein S1 specifically comprises:
inoculating the Trichoderma into a potato dextrose agar (PDA) medium, cultivating the PDA medium inoculated with the Trichoderma at 28° C. for 5 d, punching the PDA medium inoculated with the Trichoderma with a 5 mm puncher to obtain a resulting Trichoderma disc, inoculating the resulting Trichoderma disc into a potato dextrose (PD) medium, and cultivating the resulting Trichoderma disc in the PD medium at 28° C. for 3 d to obtain a resulting Trichoderma seed culture; inoculating the resulting Trichoderma seed culture into a co-culture medium in a fermentation tank, and conducting A fermentation for 35 h to 45 h; inoculating the Bacillus into a lysogeny broth agar (LBA) medium, cultivating the LBA medium inoculated with the Bacillus at 30° C. for 2 d, punching the LBA medium inoculated with the Bacillus with the 5 mm puncher to obtain a resulting Bacillus disc, inoculating the resulting Bacillus disc into a lysogeny broth (LB) medium, and cultivating the resulting Bacillus disc in the LB medium for 2 d to obtain a resulting Bacillus seed culture; and inoculating the resulting Bacillus seed culture into the co-culture medium with a Trichoderma fermentation broth, and further conducting the fermentation for 45 h to 50 h.

9. The preparation method of the biological SCA according to claim 8,
wherein the co-culture medium in the fermentation tank comprises: 20 g/L of molasses, 20 g/L of a yeast extract powder, and 20 g/L of a corn flour, and a pH of the co-culture medium is adjusted with sodium hydroxide to 5.8 to 6.0.

10. The preparation method of the biological SCA according to claim 8, wherein the resulting Trichoderma seed culture is inoculated at an amount 1.0% to 2.0% of a total fermentation amount, and the resulting Bacillus seed culture is inoculated at an amount 2.0% to 3.0% of the total fermentation amount;
at an end of the fermentation, a dissolved oxygen (DO) level rebounds to 100% of a DO level at a start of the fermentation, a pH is 7.3, a rotational speed is 120 rpm, a co-fermentation time is 45 h to 50 h, a Bacillus spore formation rate is 85%, and a Trichoderma chlamydospore formation rate is 90%; and
indexes for evaluating an antagonism and a growth promotion of the biological SCA obtained through a co-fermentation mode of the Trichoderma and the Bacillus are as follows: a content of the Trichoderma chlamydospore is 1.0×10⁸ cfu/g to 2.0×10⁸ cfu/g, a content of the Bacillus spore is 3.0-10⁹ cfu/g to 5.0×10⁹ cfu/g, a content of antimicrobial peptaibol alamethicin is 0.1 μg/g to 0.5 μg/g, and a content of IAA is 2,000 μg/g to 2,500 μg/g.

11. The preparation method of the biological SCA according to claim 6, wherein in the fermentation broth obtained based on the co-cultivation of the Trichoderma and the Bacillus, a content of a marker alamethicin is 0.04 μg/L to 0.05 μg/L, a content of indoleacetic acid (IAA) is 900 μg/L to 1,000 μg/L, a content of a Trichoderma spore is 1.0×10⁸ cfu/L to 9.0×10⁸ cfu/L, and a content of a Bacillus spore is 1.0×10⁹ cfu/L to 9.0×10⁹ cfu/L.

12. The preparation method of the biological SCA according to claim 6, wherein the biological SCA is in a dosage form of a dry powder.

13. The preparation method of the biological SCA according to claim 6, wherein the biological SCA comprises: 20% w/w to 30% w/w of microbes obtained after the co-cultivation of the Trichoderma and the Bacillus, 40% w/w to 60% w/w of a diatomaceous earth, and 12% w/w to 20% w/w of brassinolide;
wherein the brassinolide is in a form of a 0.1% solution.
